SEQUENCE LISTING (11 sequences)

SEQ ID NO: 1 - DNA, 2602 bp, Artificial
  cDNA
ttcttgtgcttcttgcccatggcggcgccggcggcgggcccgaggcgggggctgggaacagctggcacccggtcggaccttggccgccaccgccccctggccctggctggccgcccgcgctcgctgcgccgaggttgccgagctcgctgggccgcgccggaaacggggcgaggcggggccgcggcaggaagtggccctgccgggcccgagcgcttccgggtcaggaggtggtgcgcctcgcgcggcagattcgaagctagggcggggcccgcgggctgaggcagcggctgtggcggcgacgctgggcgtgaggtggcggcggccgcgccctggttgggtccccactgctctcgggggcgccatggacgaggccgtgggcgacctgaagcaggcgcttccctgtgtggccgagtcgccaacggtccacgtggaggtgcatcagcgcggcagcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacggtcctcctggcactggaaaaacatccctgtgtaaagcgttagcccagaaattgacaattagactttcaagcaggtaccgatatggccaattaattgaaataaacagccacagcctcttttctaagtggttttcggaaagtggcaagctggtaaccaagatgtttcagaagattcaggatttgattgatgataaagacgccctggtgttcgtgctgattgatgaggtggagagtctcacagccgcccgaaatgcctgcagggcgggcaccgagccatcagatgccatccgcgtggtcaatgctgtcttgacccaaattgatcagattaaaaggcattccaatgttgtgattctgaccacttctaacatcaccgagaagatcgacgtggccttcgtggacagggctgacatcaagcagtacattgggccaccctctgcagcagccatcttcaaaatctacctctcttgtttggaagaactgatgaagtgtcagatcatataccctcgccagcagctgctgaccctccgagagctagagatgattggcttcattgaaaacaacgtgtcaaaattgagccttcttttgaatgacatttcaaggaagagcgagggcctcagcggccgggtcctgagaaaactcccctttctggctcatgcgctgtatgtccaggcccccaccgtcaccatagaggggttcctccaggccctgtctctggcagtggacaagcagtttgaagagagaaagaagcttgcagcttacatctgatcctgggcttccccatctggtgcttttcccatggagaacacacaaccagtaagtgaggttgccccacacagccgtctcccagggaatcccttctgcaaaccaaacgttacttagactgcaagctagaaagccaccaaggccaggctttgttaaaagaagtgtattctatttatgttgttttaaaatgcatactgagagacaaacatcttgtcattttcactgtttgtaaaagataattcagattgtttgtctccttgtgaagaaccatcgaaacctgtttgttcccagcccacccccagtggatgggatgcataatgccagcaagttttgtttaacagcaaaaaaggaagattaatgcaggtgttatagaagccagaagagaaactgtgtcaccctaaagaagcatataatcatagcattaaaaatgcacacattactccaggtggaaggtggcaattgctttctgatatcagctcgtttgatttagtgcaaaaatgttttcaagactatttaatggatgtaaaaaagcctatttctacattataccaactgagaaaaaaatggtcggtaaagtgttctttcataataaataatcagacatggtcccatttgcaggaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 2 - DNA, 2034 bp, Artificial
  cDNA
ttcttgtgcttcttgcccatggcggcgccggcggcgggcccgaggcgggggctgggaacagctggcacccggtcggaccttggccgccaccgccccctggccctggctggccgcccgcgctcgctgcgccgaggttgccgagctcgctgggccgcgccggaaacggggcgaggcggggccgcggcaggaagtggccctgccgggcccgagcgcttccgggtcaggaggtggtgcgcctcgcgcggcagattcgaagctagggcggggcccgcgggctgaggcagcggctgtggcggcgacgctgggcgtgaggtggcggcggccgcgccctggttgggtccccactgctctcgggggcgccatggacgaggccgtgggcgacctgaagcaggcgcttccctgtgtggccgagtcgccaacggtccacgtggaggtgcatcagcgcggcagcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacggtcctcctggcactggaaaaacatccctgtgtaaagcgttagcccagaaattgacaattagactttcaagcaggtaccgatatggccaattaattgaaataaacagccacagcctcttttctaagtggttttcggaaagtggcaagctggtaaccaagatgtttcagaagattcaggatttgattgatgataaagacgccctggtgttcgtgctgattgatgaggtggagagtctcacagccgcccgaaatgcctgcagggcgggcaccgagccatcagatgccatccgcgtggtcaatgctgtcttgacccaaattgatcagattaaaaggcattccaatgttgtgattctgaccacttctaacatcaccgagaagatcgacgtggccttcgtggacagggctgacatcaagcagtacattgggccaccctctgcagcagccatcttcaaaatctacctctcttgtttggaagaactgatgaagtgtcagatcatataccctcgccagcagctgctgaccctccgagagctagagatgattggcttcattgaaaacaacgtgtcaaaattgagccttcttttgaatgacatttcaaggaagagcgagggcctcagcggccgggtcctgagaaaactcccctttctggctcatgcgctgtatgtccaggcccccaccgtcaccatagaggggttcctccaggccctgtctctggcagtggacaagcagtttgaagagagaaagaagcttgcagcttacatctgatcctgggcttccccatctggtgcttttcccatggagaacacacaaccgaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 3 - DNA, 1908 bp, Artificial
  cDNA
ttcttgtgcttcttgcccatggcggcgccggcggcgggcccgaggcgggggctgggaacagctggcacccggtcggaccttggccgccaccgccccctggccctggctggccgcccgcgctcgctgcgccgaggttgccgagctcgctgggccgcgccggaaacggggcgaggcggggccgcggcaggaagtggccctgccgggcccgagcgcttccgggtcaggaggtggtgcgcctcgcgcggcagattcgaagctagggcggggcccgcgggctgaggcagcggctgtggcggcgacgctgggcgtgaggtggcggcggccgcgccctggttgggtccccactgctctcgggggcgccatggacgaggccgtgggcgacctgaagcaggcgcttccctgtgtggccgagtcgccaacggtccacgtggaggtgcatcagcgcggcagcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacgcccccaccgtcaccatagaggggttcctccaggccctgtctctggcagtggacaagcagtttgaagagagaaagaagcttgcagcttacatctgatcctgggcttccccatctggtgcttttcccatggagaacacacaaccagtaagtgaggttgccccacacagccgtctcccagggaatcccttctgcaaaccaaacgttacttagactgcaagctagaaagccaccaaggccaggctttgttaaaagaagtgtattctatttatgttgttttaaaatgcatactgagagacaaacatcttgtcattttcactgtttgtaaaagataattcagattgtttgtctccttgtgaagaaccatcgaaacctgtttgttcccagcccacccccagtggatgggatgcataatgccagcaagttttgtttaacagcaaaaaaggaagattaatgcaggtgttatagaagccagaagagaaactgtgtcaccctaaagaagcatataatcatagcattaaaaatgcacacattactccaggtggaaggtggcaattgctttctgatatcagctcgtttgatttagtgcaaaaatgttttcaagactatttaatggatgtaaaaaagcctatttctacattataccaactgagaaaaaaatggtcggtaaagtgttctttcataataaataatcagacatggtcccatttgcaggaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 4 - DNA, 2538 bp, Artificial
  cDNA
ttcttgtgcttcttgcccatggcggcgccggcggcgggcccgaggcgggggctgggaacagctggcacccggtcggaccttggccgccaccgccccctggccctggctggccgcccgcgctcgctgcgccgaggttgccgagctcgctgggccgcgccggaaacggggcgaggcggggccgcggcaggaagtggccctgccgggcccgagcgcttccgggtcaggaggtggtgcgcctcgcgcggcagattcgaagctagggcggggcccgcgggctgaggcagcggctgtggcggcgacgctgggcgtgaggtggcggcggccgcgccctggttgggtccccactgctctcgggggcgccatggacgaggccgtgggcgacctgaagcaggcgcttccctgtgtggccgagtcgccaacggtccacgtggaggtgcatcagcgcggcagcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagcttggtatacgatgtggaagtcaaatcccatctcctcgattatgtgatgacaactttactgttttcagacaagaacgtcaacagcaacctcatcacctggaaccgggtggtgctgctccacggtcctcctggcactggaaaaacatccctgtgtaaagcgttagcccagaaattgacaattagactttcaagcaggtaccgatatggccaattaattgaaataaacagccacagcctcttttctaagtggttttcggaaagtggcaagctggtaaccaagatgtttcagaagattcaggatttgattgatgataaagacgccctggtgttcgtgctgattgatgaggtggagagtctcacagccgcccgaaatgcctgcagggcgggcaccgagccatcagatgccatccgcgtggtcaatgctgtcttgacccaaattgatcagattaaaaggcattccaatgttgtgattctgaccacttctaacatcaccgagaagatcgacgtggccttcgtggacagggctgacatcaagcagtacattgggccaccctctgcagcagccatcttcaaaatctacctctcttgtttggaagaactgatgaagtgtcagatcatataccctcgccagcagctgctgaccctccgagagctagagatgattggcttcattgaaaacaacgtgtcaaaattgagccttcttttgaatgacatttcaaggaagagcgagggcctcagcggccgggtcctgagaaaactcccctttctggctcatgcgctgtatgtccaggcccccaccgtcaccatagaggggttcctccaggccctgtctctggcagtggacaagcagtttgaagagagaaagaagcttgcagcttacatctgatcctgggcttccccatctggtgcttttcccatggagaacacacaaccagtaagtgaggttgccccacacagccgtctcccagggaatcccttctgcaaaccaaacgttacttagactgcaagctagaaagccaccaaggccaggctttgttaaaagaagtgtattctatttatgttgttttaaaatgcatactgagagacaaacatcttgtcattttcactgtttgtaaaagataattcagattgtttgtctccttgtgaagaaccatcgaaacctgtttgttcccagcccacccccagtggatgggatgcataatgccagcaagttttgtttaacagcaaaaaaggaagattaatgcaggtgttatagaagccagaagagaaactgtgtcaccctaaagaagcatataatcatagcattaaaaatgcacacattactccaggtggaaggtggcaattgctttctgatatcagctcgtttgatttagtgcaaaaatgttttcaagactatttaatggatgtaaaaaagcctatttctacattataccaactgagaaaaaaatggtcggtaaagtgttctttcataataaataatcagacatggtcccatttgcaggaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 5 - DNA, 2240 bp, Artificial
  cDNA
ctgtgctgctccctggagtggggggaactcagcggcggggccagaccttcacagaccctccttatgtccacggagggtggattgaggtcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacggtcctcctggcactggaaaaacatccctgtgtaaagcgttagcccagaaattgacaattagactttcaagcaggtaccgatatggccaattaattgaaataaacagccacagcctcttttctaagtggttttcggaaagtggcaagctggtaaccaagatgtttcagaagattcaggatttgattgatgataaagacgccctggtgttcgtgctgattgatgaggtggagagtctcacagccgcccgaaatgcctgcagggcgggcaccgagccatcagatgccatccgcgtggtcaatgctgtcttgacccaaattgatcagattaaaaggcattccaatgttgtgattctgaccacttctaacatcaccgagaagatcgacgtggccttcgtggacagggctgacatcaagcagtacattgggccaccctctgcagcagccatcttcaaaatctacctctcttgtttggaagaactgatgaagtgtcagatcatataccctcgccagcagctgctgaccctccgagagctagagatgattggcttcattgaaaacaacgtgtcaaaattgagccttcttttgaatgacatttcaaggaagagcgagggcctcagcggccgggtcctgagaaaactcccctttctggctcatgcgctgtatgtccaggcccccaccgtcaccatagaggggttcctccaggccctgtctctggcagtggacaagcagtttgaagagagaaagaagcttgcagcttacatctgatcctgggcttccccatctggtgcttttcccatggagaacacacaaccagtaagtgaggttgccccacacagccgtctcccagggaatcccttctgcaaaccaaacgttacttagactgcaagctagaaagccaccaaggccaggctttgttaaaagaagtgtattctatttatgttgttttaaaatgcatactgagagacaaacatcttgtcattttcactgtttgtaaaagataattcagattgtttgtctccttgtgaagaaccatcgaaacctgtttgttcccagcccacccccagtggatgggatgcataatgccagcaagttttgtttaacagcaaaaaaggaagattaatgcaggtgttatagaagccagaagagaaactgtgtcaccctaaagaagcatataatcatagcattaaaaatgcacacattactccaggtggaaggtggcaattgctttctgatatcagctcgtttgatttagtgcaaaaatgttttcaagactatttaatggatgtaaaaaagcctatttctacattataccaactgagaaaaaaatggtcggtaaagtgttctttcataataaataatcagacatggtcccatttgcaggaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 6 - DNA, 1672 bp, Artificial
  cDNA
ctgtgctgctccctggagtggggggaactcagcggcggggccagaccttcacagaccctccttatgtccacggagggtggattgaggtcagcactgcaaagaaagaagacataaacctgagtgttagaaagctactcaacagacataatattgtgtttggtgattacacatggactgagtttgatgaaccttttttgaccagaaatgtgcagtctgtgtctattattgacacagaattaaaggttaaagactcacagcccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacggtcctcctggcactggaaaaacatccctgtgtaaagcgttagcccagaaattgacaattagactttcaagcaggtaccgatatggccaattaattgaaataaacagccacagcctcttttctaagtggttttcggaaagtggcaagctggtaaccaagatgtttcagaagattcaggatttgattgatgataaagacgccctggtgttcgtgctgattgatgaggtggagagtctcacagccgcccgaaatgcctgcagggcgggcaccgagccatcagatgccatccgcgtggtcaatgctgtcttgacccaaattgatcagattaaaaggcattccaatgttgtgattctgaccacttctaacatcaccgagaagatcgacgtggccttcgtggacagggctgacatcaagcagtacattgggccaccctctgcagcagccatcttcaaaatctacctctcttgtttggaagaactgatgaagtgtcagatcatataccctcgccagcagctgctgaccctccgagagctagagatgattggcttcattgaaaacaacgtgtcaaaattgagccttcttttgaatgacatttcaaggaagagcgagggcctcagcggccgggtcctgagaaaactcccctttctggctcatgcgctgtatgtccaggcccccaccgtcaccatagaggggttcctccaggccctgtctctggcagtggacaagcagtttgaagagagaaagaagcttgcagcttacatctgatcctgggcttccccatctggtgcttttcccatggagaacacacaaccgaaaagtgcagactctgagtgttccagggaaacacatgctggacatcccttgtaacccggtatgggcgcccctgcattgctgggatgtttctgcccacggttttgtttgtgcaataacgttatcacatttctaatgaggattcacattaatataatataaaataaataggtcagttactggtctctttctccgaatgttatgttttgcttttatctcacagtaaaataaatataattaatggtttgcatgtgaaattcacttttgaaagaacatgttaccttaccttttgttttagaagttttcaagtattaaaatattttttagaa

SEQ ID NO: 7 - protein, 432 aa, Artificial
  Putative Protein Derived from cDNA.
MDEAVGDLKQALPCVAESPTVHVEVHQRGSSTAKKEDINLSVRKLLNRHNIVFGDYTWTEFDEPFLTRNVQSVSIIDTELKVKDSQPIDLSACTVALHIFQLNEDGPSSENLEEETENIIAANHWVLPAAEFHGLWDSLVYDVEVKSHLLDYVMTTLLFSDKNVNSNLITWNRVVLLHGPPGTGKTSLCKALAQKLTIRLSSRYRYGQLIEINSHSLFSKWFSESGKLVTKMFQKIQDLIDDKDALVFVLIDEVESLTAARNACRAGTEPSDAIRVVNAVLTQIDQIKRHSNVVILTTSNITEKIDVAFVDRADIKQYIGPPSAAAIFKIYLSCLEELMKCQIIYPRQQLLTLRELEMIGFIENNVSKLSLLLNDISRKSEGLSGRVLRKLPFLAHALYVQAPTVTIEGFLQALSLAVDKQFEERKKLAAYI

SEQ ID NO: 8 - protein, 552 aa, Artificial
  Putative Protein Derived from cDNA.
SCASCPWRRRRRARGGGWEQLAPGRTLAATAPWPWLAARARCAEVAELAGPRRKRGEAGPRQEVALPGPSASGSGGGAPRAADSKLGRGPRAEAAAVAATLGVRWRRPRPGWVPTALGGAMDEAVGDLKQALPCVAESPTVHVEVHQRGSSTAKKEDINLSVRKLLNRHNIVFGDYTWTEFDEPFLTRNVQSVSIIDTELKVKDSQPIDLSACTVALHIFQLNEDGPSSENLEEETENIIAANHWVLPAAEFHGLWDSLVYDVEVKSHLLDYVMTTLLFSDKNVNSNLITWNRVVLLHGPPGTGKTSLCKALAQKLTIRLSSRYRYGQLIEINSHSLFSKWFSESGKLVTKMFQKIQDLIDDKDALVFVLIDEVESLTAARNACRAGTEPSDAIRVVNAVLTQIDQIKRHSNVVILTTSNITEKIDVAFVDRADIKQYIGPPSAAAIFKIYLSCLEELMKCQIIYPRQQLLTLRELEMIGFIENNVSKLSLLLNDISRKSEGLSGRVLRKLPFLAHALYVQAPTVTIEGFLQALSLAVDKQFEERKKLAAYI

SEQ ID NO: 9 - protein, 295 aa, Artificial
  Putative Protein Derived from cDNA.
SCASCPWRRRRRARGGGWEQLAPGRTLAATAPWPWLAARARCAEVAELAGPRRKRGEAGPRQEVALPGPSASGSGGGAPRAADSKLGRGPRAEAAAVAATLGVRWRRPRPGWVPTALGGAMDEAVGDLKQALPCVAESPTVHVEVHQRGSSTAKKEDINLSVRKLLNRHNIVFGDYTWTEFDEPFLTRNVQSVSIIDTELKVKDSQPIDLSACTVALHIFQLNEDGPSSENLEEETENIIAANHWVLPAAEFHGLWDSLVYDVEVKSHLLDYVMTTLLFSDKNVNSNLITPPPSP

SEQ ID NO: 10 - protein, 279 aa, Artificial
  Putative Protein Derived from cDNA.
MTTLLFSDKNVNSNLITWNRVVLLHGPPGTGKTSLCKALAQKLTIRLSSRYRYGQLIEINSHSLFSKWFSESGKLVTKMFQKIQDLIDDKDALVFVLIDEVESLTAARNACRAGTEPSDAIRVVNAVLTQIDQIKRHSNVVILTTSNITEKIDVAFVDRADIKQYIGPPSAAAIFKIYLSCLEELMKCQIIYPRQQLLTLRELEMIGFIENNVSKLSLLLNDISRKSEGLSGRVLRKLPFLAHALYVQAPTVTIEGFLQALSLAVDKQFEERKKLAAYI

SEQ ID NO: 11 - protein, 431 aa, Artificial
  Putative Protein Derived from cDNA.
VLLPGVGGTQRRGQTFTDPPYVHGGWIEVSTAKKEDINLSVRKLLNRHNIVFGDYTWTEFDEPFLTRNVQSVSIIDTELKVKDSQPIDLSACTVALHIFQLNEDGPSSENLEEETENIIAANHWVLPAAEFHGLWDSLVYDVEVKSHLLDYVMTTLLFSDKNVNSNLITWNRVVLLHGPPGTGKTSLCKALAQKLTIRLSSRYRYGQLIEINSHSLFSKWFSESGKLVTKMFQKIQDLIDDKDALVFVLIDEVESLTAARNACRAGTEPSDAIRVVNAVLTQIDQIKRHSNVVILTTSNITEKIDVAFVDRADIKQYIGPPSAAAIFKIYLSCLEELMKCQIIYPRQQLLTLRELEMIGFIENNVSKLSLLLNDISRKSEGLSGRVLRKLPFLAHALYVQAPTVTIEGFLQALSLAVDKQFEERKKLAAYI

(19) United States
(12) Patent Application Publication
(10) Pub. No.: US 2006/0134622 A1
(43) Pub. Date: Jun. 22, 2006

(54) Amplified cancer target genes useful in diagnosis and thereapeutic screening

(76) Inventors: Meena Augustus, Gaithersburg, MD (US); Stephen Horrigan, Gaithersburg, MD (US); Colyn Cain, Bethesda, MD (US); Jeffrey W Strovel, Laurel, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 10/540,311

(22) PCT Filed: Dec. 19, 2003
(86) PCT No.: PCT/US03/40701

§ 371 Date: Dec. 12, 2005

Related U.S. Application Data
(60) Provisional application No. 60/434,918, filed on Dec. 20, 2002.
(60) Provisional application No. 60/463,577, filed on Apr. 17, 2003.

(51) Int. Cl. C12Q 1/68

(52) U.S. Cl. 435/6

(57) ABSTRACT

A gene useful as a prognostic indicator of survival of patients afflicted with cancers such as breast cancer is described. Also disclosed are methods for screening potential therapeutic agents, such as antitumor agents, based on their modulation of the expression of a TRIP13 gene, or splice variants thereof, along with methods for diagnosing cancerous, or potentially cancerous, conditions as a result of the expression, or patterns of expression, of such genes, including changes in copy number. Also disclosed are methods for detecting or determining functionally related genes, where disruption of TRIP13 may affect such related-gene expression, as well as methods for treating cancer based on targeting expression products of such genes and determining genes involved in the cancerous process. Methods for identifying, detecting and following patients and others having high levels of amplification/expression of the gene before treatment where differences after treatment serve as markers for indication of success of treatment or for identifying individuals who would benefit from such treatment using this gene marker as a diagnostic and/or prognostic and/or pharmacodynamic or surrogate marker are described.

This application claims priority of U.S. Provisional Application Ser. No. 60/434,918, filed 20 Dec. 2002, and 60/463,577, filed 17 Apr. 2003, the disclosures of which are hereby incorporated by reference in their entirety.

### FIELD OF THE INVENTION

The present invention relates to a gene amplified and transcriptionally over-expressed in cancer, including RNA splice variants thereof, along with putative polypeptides encoded by said splice variants, for use in diagnosis of cancerous conditions as well as therapeutic screening for anti-neoplastic agents.

### BACKGROUND OF THE INVENTION

Screening assays for novel drugs are based on the response of model cell based systems in vitro to treatment with specific compounds. Various measures of cellular response have been utilized, including the release of cytokines, alterations in cell surface markers, activation of specific enzymes, as well as alterations in ion flux and/or pH. Some such screens rely on specific genes, such as oncogenes (or gene mutations).

In addition, chromosomal abnormalities have been identified in most cancer cells. Conventional chromosome banding techniques allow for the detection of specific chromosomal defects in tumor cells but interpretation of the banding pattern is sometimes difficult, particularly when complex chromosomal rearrangements or subtle abnormalities are present. In recent years, new techniques, such as CGH and SKY, based on fluorescent in situ hybridization (FISH) (Pinkel D, Segraves R, Sudar D, Clark S, Poole I, Kowbel D, Collins C, Kuo W L, Chen C, Zhai Y, Dairkee S H, Ljung B M, Gray J W, Albertson D G. High resolution analysis of DNA copy number variation using comparative genomic hybridization to microarrays. Nat Genet. 1998 October;20(2):207-1 1. have been developed to overcome the limitations of conventional chromosome banding. CGH measures intensities of fluorescently labeled tumor DNA and normal DNA following hybridization to normal chromosomes Kallioniemi A, Kallioniemi O P, Sudar D, Rutovitz D, Gray J W, Waldman F, Pinkel D. Comparative genomic hybridization for molecular cytogenetic analysis of solid tumors. Science. 1992 Oct. 30;258(5083):818-21. Gain or loss of copy number of a particular chromosome or chromosome region in DNA, such as tumor DNA, is determined by the relative intensity of a fluorescence ratio. SKY utilizes a cocktail of chromosome probes, fluorescently labeled to specify each chromosome, which is hybridized to tumor chromosomes in an effort to identify numerical and structural abnormalities in the tumor cell (Schrock E, du Manoir S, Veldman T, Schoell B, Wienberg J, Ferguson-Smith M A, Ning Y, Ledbefter D H, Bar-Am I, Soenksen D, Garini Y, Ried T. Multicolor spectral karyotyping of human chromosomes. Science. 1996 Jul. 26;273(5274):494-7. CGH and SKY have been used to identify chromosomal regions that harbor genes significant to the process of tumor initiation or progression.

Thus, increase in copy number indicates genomic amplification whereas increased levels of messenger RNA indicates over-expression of a gene (at least at the transcriptional level), and both can be important in the onset and/or progression of cancer, such as the development of metastasis. In accordance with the present invention, a gene, called TRIP13 (Thyroid hormone Receptor Interacting Protein), has been identified that is both amplified and transcriptionally over-expressed in tumor cells but not in otherwise normal tissues.

The thyroid hormone (T3) receptors (TRs) are hormone-dependent transcription factors that regulate expression of a variety of specific target genes. Lee et al. (Two classes of proteins dependent on either the presence or absence of thyroid hormone for interaction with the thyroid hormone receptor. Mol Endocrinol 9(2):243-54 (1995)) isolated clones encoding proteins that specifically interact with the ligand binding domain of the rat TR beta and several such proteins, were isolated from independent selections carried out either in the presence or absence of T3. Surprisingly, all of the Trips were dependent on hormone for interaction with the TR, with some interacting only when T3 is present and others only when it is absent. Nearly all of the Trips also show similar ligand-dependent interaction with the retinoid X receptor (RXR), but none interact with the glucocorticoid receptor under any conditions. Trips have inherent transcriptional activity. However, TRIPs have not been implicated in the cancerous process.

Genomic amplification is an established mechanism for increasing the expression of genes involved in the initiation and progression of cancer. Because of their high level of expression, proteins encoded by such genes are prime molecular targets for anti-cancer therapies. The present invention takes advantages of these techniques by providing a system that integrates high-resolution cytogenetic and molecular maps with global expression profiling. High-resolution comparative genomic hybridization (CGH), array CGH, and whole-genome expression analysis were applied to rapidly identify highly amplified over-expressed candidate oncogenes. Quantitative PCR then confirmed DNA copy number and mRNA expression levels in relevant tumor cell lines and resulted in the identification of the thyroid hormone receptor interacting protein 13 gene, TRIP13, in chromosomal region 5p15 (which was found to be both amplified and over-expressed in a breast cancer cell line).

Two functions for the TRIP13 protein are known. It interacts with the ligand binding domain of thyroid hormone receptor, and with the human papillomavirus type 16 (HPV16) E1 protein. It contains a single known protein domain termed an AAA domain (this refers to an ATPase family associated with various cellular activities). This domain is shared by a large family of proteins that regulate and are thought to perform chaperone-like functions that assist in the assembly, operation, or disassembly of protein complexes. Protein homology searches indicate it is a member of a subfamily of AM domain containing proteins which include the Pachytene Checkpoint Protein 2 (Pch2), Cell Division Control Protein 48 (Cdo48). These are a family of proteins involved in the regulation of the cell cycle.

### BRIEF SUMMARY OF THE INVENTION

In one aspect, the present invention relates to a method for identifying a gene modulating agent, comprising determining the ability of said compound to modulate the activity of a cancer-related gene as disclosed herein. Such modulation may take the form of modulating gene expression, polypeptide synthesis or enzyme activity. In a preferred embodiment, the change in expression is a decrease in expression, such as where the decrease in expression is a decrease in copy number of the gene.

In other preferred embodiments of such a screening process, the change in expression is a decrease in the synthesis of an RNA encoded by said gene or a decrease in the synthesis of a polypeptide encoded by said gene.

In a further aspect, the present invention relates to a method for identifying an anti-neoplastic agent comprising contacting a cancerous cell with a compound found to have gene modulating activity in one or more of the screening methods of the invention under conditions promoting the growth of said cell and detecting a change in the activity of said cancerous cell. In all such methods, the cell may be a recombinant cell.

In another aspect, the present invention relates to a method for diagnosing the presence of a cancerous condition, or diagnosing a predisposition to developing a cancerous condition, in an animal, especially a human being, by determining the amplification and/or over-expression, of one or more genes as disclosed herein.

In a further aspect, the present invention relates to a method for the treatment of a cancerous condition, especially one involving breast, colon, lung or prostate tissues, especially breast, or any solid tumor, utilizing selected chemical agents having antitumor activity as identified using one of the assays disclosed herein.

In a still further aspect, the present invention relates to a method for detecting or determining a cancer initiating, facilitating or suppressing gene comprising contacting a cancerous cell with an agent that modulates the activity of a gene as disclosed herein and determining a change in activity of said gene(s).

In another embodiment, the present invention provides a method for monitoring the progress of a cancer treatment, such as where the methods of the invention permit a determination that a given course of cancer therapy is or is not proving effective because of an increased or decreased expression of a gene, or genes, disclosed herein.

In a further aspect, the present invention relates to methods for identifying, detecting and following patients and others having high levels of amplification/expression of the gene before treatment where differences after treatment serve as markers for indication of success of treatment.

### BRIEF DESCRIPTION OF THE DRAWING

FIG. 1 shows Kaplan-Meier survival analysis results based on BAC probe amplification in breast cancer specimens. Panel B shows amplified versus normal while panel A shows high and low level amplification versus normal tissue as a function of survival rate. Here, relative level of amplification is shown at the ordinate while months of survival of the patient from whom the specimen was retrieved is shown at the abscissa and indicates a significant association of TRIP13 amplification with poor survival.

### DEFINITIONS

As used herein, the following terms have the indicated meaning unless expressly stated otherwise.

The term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases, or any stretch of polynucleotides that could be synthetically synthesized.

As used herein, the term “DNA segment” or “DNA sequence” refers to a DNA polymer, in the form of a separate fragment or as a component of a larger DNA construct, which has been derived from DNA, and may include both single stranded and duplex sequences. Such segments are provided in the form of an open reading frame uninterrupted by internal non-translated sequences, or introns, which are typically present in eukaryotic genes.

The term “coding region” refers to that portion of a gene which either naturally or normally codes for the expression product of that gene in its natural genomic environment, i.e., the region coding in vivo for the native expression product of the gene.

the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s). This term may also be applied to an RNA species transcribed from a gene.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “fragment,” when referring to a coding sequence, means a portion of DNA comprising less than the complete coding region whose expression product retains essentially the same biological function or activity as the expression product of the complete coding region.

The terms “antibody” and “immunoglobulin” are considered herein as interchangeable. With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, such as cows, goats and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al, J. Biol. Chem. 262:6609-6616 (1977).

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors.

As used herein, the term “biological activity” refers to any measurable chemical activity of a polypeptide product encoded by TRIP13 wherein said activity can be quantitatively measured and wherein linked to the cancerous state so that inhibition of such biological activity also results in reduction of cancerous growth or other cancer-related activity in a cell. In terms of the present invention, this includes activities such as, but not limited to, activity dependent upon the AM domain of TRIP13 polypeptide as well as binding to thyroid hormone.

As used herein, the term “test compound” means a chemical compound, such as a small organic compound, that can be screened for activity in any of the assays of the invention, such as modulating expression of the TRIP13 gene or modulation of a biological activity of TRIP13 protein or polypeptide. The term “agent” is used interchangeably with the term “compound” and likewise the term “test agent” is used interchangeably with the term “test compound.”

### DETAILED SUMMARY OF THE INVENTION

In one aspect the present invention relates to a gene that corresponds to a polynucleotide comprising a nucleotide sequence of SEQ ID NO: 1-6, each sequence representing variants in sequence and the exons present, and found to be amplified and over-expressed in cancerous tissues. Gene sequences that demonstrate amplification and/or over-expression are indicative of the cancerous status of a given cell. More particularly, such genes when amplified and/or over-expressed in cancerous tissues, as compared to non-cancerous cells, from a specific organ are genes that correspond to a polynucleotide comprising a nucleotide sequence of SEQ ID NO: 1-6. Polypeptides with amino acid sequences encoded by these nucleotide sequences are shown as SEQ ID NO: 6 -8. Here, the polypeptide of SEQ ID NO: 7 is encoded by the nucleotide sequence of the cDNA of SEQ ID NO: 1, the polypeptide of SEQ ID NO: 8 is encoded by the nucleotide sequence of the cDNA of SEQ ID NO: 2, the polypeptide of SEQ ID NO: 9 is encoded by the nucleotide sequence of the cDNA of SEQ ID NO: 3, the polypeptide of SEQ ID NO: 10 is encoded by the nucleotide sequence of the cDNA of SEQ ID NO: 4, and the polypeptide of SEQ ID NO: 11 is encoded by the nucleotide sequence of the cDNA of SEQ ID NO: 5 and/or 6.

The present invention utilized expression analysis of TRIP13 in a large sub-set of clinical tumor samples indicated that it is over-expressed in about 40% of epithelial tumors. In situ hybridization and immunohistochemistry showed high-level expression of RNA and protein in the epithelial cells of human tumors. The predicted protein sequence contains an ATPase domain and has similarity to proteins involved in cell cycle checkpoint control. Expression analysis of a large number of clinical tumor samples shows significant correlation with expression levels of genes involved in chromosome maintenance and segregation. Decrease in the mRNA level of TRIP13 by RNA interference resulted in mitotic arrest in breast cancer cells. The data suggests that this gene is involved in mitotic checkpoint control of cancer cells. The novel disclosure thereby provides a target discovery pipeline for the rapid identification of drug targets.

In a preferred embodiment, where disruption of TRIP13 affects expression of other genes, especially structurally and/or functionally related genes, the present invention encompasses use of such other genes, and their affected expression, as a means of aiding the cancer diagnostic and/or treatment processes.

In accordance with the foregoing, the present invention relates to a gene, dubbed TRIP13, that is amplified at the genomic level and over-expressed transcriptionally in cancer. Further, such gene finds use inter alia as a diagnostic marker for tumor state, stage and grade, for use as a prognostic marker to predict response to therapy or response to specific therapy, for use as a target for therapeutic molecule, such as an antibody or small molecule (which could be used to either direct the therapy to the tumor cell or to inhibit the activity of the protein to disrupt the tumor cell function) and as a marker for screening for drug activity based on the activity of the protein, transcriptional state of the gene, or the transcriptional state of target genes activated by TRIP13.

The characteristics of TRIP13 have been identified through a combination of CGH, SKY, mRNA expression analysis, quantitative Polymerase Chain Reaction and Reverse Transcriptase-Polymerase Chain Reaction (RT-PCR). Such genes are both markers and potential therapeutic targets for cancer, in preferably breast, colon, lung and prostate malignancies, most preferably breast. In addition, the amplified nature of such genes provides a means of diagnosing a cancerous condition, or predisposition to a cancerous conditions, by determining the amplification of one or more of such genes in a patient afflicted with, or predisposed toward, or otherwise at risk of developing, cancer. TRIP13 is found as a number of splice variants, represented by the cDNA sequences of SEQ ID NO: 1-6.

The procedures used to identify the genes disclosed herein may be summarized as follows:

For CGH analysis, based on detailed molecular cytogenetic characterizations, data sets are generated that may include regions reported in the public domain as well as unique regions not previously known. In general, a map of chromosomal regions involved in consistent, recurrent and high level genomic gains (i.e., amplifications) for a representative cancer cell line or tumor type (e.g. colon, prostate, breast and lung) that can be recognized as a pattern/signature for a tumor is assembled. (A map of chromosomal regions containing genomic losses (i.e., deletions) in tumor cells, such as for an individual cell line to be examined may likewise be generated). Levels of intensities of gains and losses are categorized for entry into a database. A comparison of the patterns of gains and losses between the clinical samples (e.g. colon xenografts) and cell lines (e.g., colon) of matched Stages and Grades is then produced. In addition, this facilitates comparison of the patterns of gains and losses between primary tumor cell lines and metastatic prostate tumor cell lines.

In accordance with the present invention, for SKY analysis data sets were generated by identification and development of a database of novel chromosomal rearrangements in cancer cell lines, identification of novel translocations involving specific chromosomes or chromosomal regions and followed by reconciliation of SKY and CGH analysis on the same cell line or cell type as a verification of the combined findings.

Combining data from the genomic DNA analysis of gains in the tumor cell lines/clinical samples with mRNA expression analysis from the same and matched tumor types displayed on an assembled human genome seqeunce obtained from the NCBI Genbank seqeuence repository.:
1. Regions of genomic amplification were identified in tumor cell lines and clinical tumor samples using comparative genomic hybridization.
2. The assembled human genomic sequence was used identify the DNA sequences that are present in the amplified region
3. Quantitative PCR on genomic DNA derived from tumor cell lines and clinical tumor samples was used to identify the genomic region with the highest amplification levels
4. The assembled human genomic sequence was used to identify the DNA sequences present in the regions of amplification. All putative genes within this region of genomic DNA were identified.
5. For all putative genes within the amplified region genomic copy number status was identified by quantitative PCR and mRNA expression levels were identified using quantitative RT-PCR.
6. For the genomic region with the highest DNA copy number amplification a Bacterial artificial chromosome (BAC) was identified that contained approximately 100 kb of human genomic sequence identical to this region. This BAC was used to confirm the genomic amplification by FISH in tumor cell lines and clinical tissue samples.
7. SKY analysis was done on tumor cell lines with the amplification to determine the mechanism of DNA copy number increase.
8. The gene that were consistantly amplified at the genomic level and overexpressed at the mRNA level are further characterized for function (i.e by protein expression, RNA interference).

In accordance with the present invention, over-expression of cellular genes is conveniently monitored in model cellular systems using cell lines (such as is used in the example below), primary cells, or tissue samples maintained in growth media. For different purposes, these may be treated with compounds at one or more different concentrations to assay for modulating agents. Thus, cellular RNAs were isolated from the cells or cultures as an indicator of selected gene expression. The cellular RNAs were then divided and subjected to analysis that detected the presence and/or quantity of specific RNA transcripts, which transcripts were then amplified for detection purposes using standard methodologies, such as reverse transcriptase polymerase chain reaction (RT-PCR). The levels of specific RNA transcripts, including their presence or absence, were determined. When used for identification of modulating agents, such as anti-neoplastic agents, a metric is derived for the type and degree of response of the treated sample compared to control samples.

In accordance with the foregoing, the TRIP13 gene was identified as being amplified and over-expressed, which included increased copy number thereof, in cancerous cells In particular, such gene includes genes that correspond to a polynucleotide comprising a splice variant having the nucleotide sequence of SEQ ID NO: 1-6, especially where it comprises one of these sequences.

This gene may be utilized to characterize the cancerous, or non-cancerous, status of cells, or tissues. The methods of the invention may be used with a variety of cell lines or with primary samples from tumors maintained in vitro under suitable culture conditions for varying periods of time, or in situ in suitable animal models.

The gene disclosed herein is expressed at levels in cancer cells that are different from the expression levels in non-cancer cells. The splice variants of this gene (SEQ ID NO: 1-6 as the corresponding cDNA sequences) are amplified in cancer cells relative to non-cancer cells of corresponding tissues.

In one aspect, the present invention relates to a method for identifying a TRIP13 gene modulating agent, comprising:

(a) contacting a test compound with a cell that expresses a TRIP 13 gene; and

(b) determining a change in expression of said gene as a result of said contacting, wherein a change in said determined expression indicates gene modulation,

thereby identifying said test compound as a gene modulating agent.

In a more specific embodiment, the present invention also relates to a method for identifying a gene modulating agent, such as an anti-neoplastic agent, comprising:

(a) contacting a compound with a cell that expresses at least one gene corresponding to a polynucleotide that comprises a nucleotide sequence selected from SEQ ID NO: 1-6 and under conditions promoting such expression, and

(b) detecting a change in expression of said gene compared to expression when said compound is not present and/or when said contacting does not, or has not, occurred,

wherein a change in expression of said gene is indicative of anti-neoplastic activity.

Because the gene disclosed herein is over-expressed and relates to the cancerous condition of a cell, successful anti-neoplastic activity will commonly be exhibited by agents that reduce the expression of this gene (i.e., a gene that corresponds to a polynucleotide comprising the nucleotide sequences of SEQ ID NO: 1-6, wherein the latter are cDNA sequences identified from the corresponding mRNA sequences and represent splice variants of TRIP13 and wherein the expression of TRIP13 or a gene corresponding to TRIP13 is being modulated by the agent whose gene-modulating activity is being determined).

In one embodiment thereof, the change in expression is a decrease in level of mRNA transcribed from the TRIP13 gene. In accordance therewith, said change in gene expression level is conveniently determined by detecting a change in expression of messenger RNA encoded by said gene sequence.

Other methods useful in measuring a change in expression of TRIP13 include measuring a change in the amount or rate of synthesis of a polypeptide encoded by said gene, preferably a decrease in synthesis of said polypeptide. Preferably, the polypeptide comprises an amino acid sequence highly homologous to a sequence of SEQ ID NO: 7-11, and most preferably wherein the polypeptide comprises such sequence.

The methods of the invention can thus be utilized to identify anti-neoplastic agents useful in treatment of cancerous conditions. Such activity can be further modified by first identifying such an agent using an assay as already described and further contacting such agent with a cancerous cell, followed by monitoring of the status of said cell, or cells. A change in status indicative of successful anti-neoplastic activity may include a decrease in the rate of replication of the cancerous cell(s), a decrease in the total number of progeny cells that can be produced by said cancerous cell(s), or a decrease in the number of times said cancerous cell(s) can replicate, or the death of said cancerous cell(s).

Anti-neoplastic agents may also be identified using recombinant cells suitably engineered to contain and express the TRIP13 gene. In one such embodiment, a recombinant cell is formed using standard technology and then utilized in the assays disclosed herein; Methods of forming such recombinant cells are well known in the literature. See, for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

In a further aspect, the present invention relates to a method for identifying an agent that modulates a TRIP13 polypeptide biological activity, comprising:

(a) contacting a test compound with a TRIP13 polypeptide; and

(b) determining a change in biological activity of said TRIP13 polypeptide as a result of said contacting, wherein a change in said biological activity indicates modulation of TRIP13, biological activity,

thereby identifying said test compound as an agent that modulates TRIP13 biological activity.

In a preferred embodiment of the foregoing, the determined change is a decrease in biological activity. In another such preferred embodiment, the TRIP13 polypeptide is present in a cell, more preferably a mammalian cell, such as where the cell has been engineered to contain a TRIP13 polypeptide. In some such embodiments, the cell does not normally contain TRIP13 protein absent such engineering.

Especially preferred is where the TRIP13 polypeptide comprises an amino acid sequence selected from SEQ ID NO: 7, 8, 9, 10, 11 and 12, which sequence may be part of a larger polypeptide. Also useful are embodiments wherein TRIP13 polypeptide is immobilized on a solid support, especially a glass or plastic support, many examples of which are well known to those skilled in the art.

The type of biological activity to be determined as a basis for determining such modulation is varied and includes any biological activity that is related to the cancerous properties of a cell as determined, induced, facilitated or supported by the expression, or presence, of TRIP13 polypeptide in the call. This may include assays based on the activity of TRIP13 in binding thyroid hormone or may involve use of thyroid hormone receptor. Such assays may also be based on the presence of the AM domain in TRIP13. Assays for binding to AM domain are well known to those of skill in the art and will not be described in detail herein. The biological activity of TRIP13 can also be monitored by determining the effects of test compounds on ATPase activity. Examples of such assays described in the literature for proteins with AM domains can be found in numerous published articles, including, but not limited to, the following: Hartman et al., Katanin, a microtubule-severing protein, is a novel AAA ATPase that targets to the centromere using a WD40-containing subunit, Cell, 93:277-287 (1998); Joshi et al., C-terminal domain mutations in ClpX uncouple substrate binding from an engagement step required for unfolding, Mol. Microbiol., 48:67-76 (2003); Corydon et al., Human and mouse mitochondrial orthologs of bacterial ClpX. Mammalian Genome, 11:899-905 (2000); and Li and Sha, Cloning, expression, purification and preliminary X-ray crystallographic studies of Escherichia coli Hsp100 nucleotide-binding domain 2 (NBD2), Acta Crystallogr D Biol Crystallogr, 58(Pt 6 Pt 2):1030-1031 (2002), the disclosures of all of which are hereby incorporated by reference in their entirety. Using such methods, entire test compound libraries can be quickly screened for modulation of TRIP13 biological activity.

The present invention also relates to a method for detecting the cancerous status of a cell, comprising detecting elevated copy number and/or expression in said cell of at least one gene that corresponds to TRIP13, in particular a gene expressing an RNA whose cDNA is a one of SEQ ID NOS: 1-6. Such elevated expression may be readily monitored by comparison to that of otherwise normal cells having the same gene. Elevated expression of this gene is indicative of the cancerous state. This includes a gene corresponding to a polynucleotide that comprises a nucleotide sequence selected from SEQ ID NO: 1-6. Such elevated expression, including increased copy number, may be the expression of more than one such gene.

The present invention also relates to a method for detecting a cancer-linked gene comprising the steps of contacting a compound identified as having gene modulating activity for a gene corresponding to a polynucleotide that comprises a nucleotide sequence selected from SEQ ID NO: 1-6 with a cell expressing a test gene and detecting modulation, such as decreased activity, of such test gene relative to when said compound is not present or when said contacting does not, or has not, occurred, thereby identifying said test gene as a cancer-related gene. In preferred embodiments, the gene determined by said process is an oncogene, or cancer-facilitating gene.

In another embodiment, there is provided a method for treating cancer comprising contacting a cancerous cell with an agent first identified as having gene modulating activity using any of the assay processes disclosed according to the invention and in an amount effective to reduce the cancerous activity of said cell. In a preferred embodiment, the cancerous cell is contacted in vivo. In other preferred embodiments, said reduction in cancerous activity is a decrease in the rate of proliferation of said cancerous cell, or said reduction in cancerous activity is the death of said cancerous cell.

The present invention further relates to a method for treating and/or diagnosing cancer comprising contacting a cancerous cell with an agent having activity against an expression product encoded by a gene corresponding to a polynucleotide comprising a nucleotide sequence selected from the group consisting of SEQ ID NO: 1-6, preferably where the expression product is a polypeptide, most preferably one comprising an amino acid sequence selected from SEQ ID NO: 7-11. In a preferred embodiment, said cancerous cell is contacted in vivo. In another preferred embodiment, the agent is an antibody.

As noted, the genes useful in the assays of the invention are genes corresponding to TRIP13, or a gene corresponding to TRIP13, or a mutated form of TRIP13, and include splice variants such as one of the polynucleotides having the sequence of SEQ ID NO: 1-6 (i.e., a gene that encodes the same RNA, such as the same messenger RNA, whose corresponding cDNA is one of the sequences of SEQ ID NO: 1-6). The genes useful in the processes of the invention further include genes encoding RNAs whose corresponding cDNA is at least 90% identical to a sequence selected from SEQ ID NO: 1-6, preferably at least about 95% identical to such a sequence, more preferably at least about 98% identical to such sequence and most preferably one comprising that sequence are specifically contemplated by all of the processes of the present invention.

In addition, sequences encoding the same proteins (SEQ ID NO: 7-11) as any of these sequences, regardless of the percent identity of such sequences, are also specifically contemplated by the invention.

The genes corresponding to TRIP13, and therefore useful in the methods of the invention, may be genomic in nature and thus represent the sequence of an actual gene, such as a human gene, or may be a cDNA sequence derived from a messenger RNA (mRNA) and thus represent contiguous exonic sequences derived from a corresponding genomic sequence or they may be wholly synthetic in origin for purposes of detecting. As described in the Example, the expression of these cancer-related genes is determined from the relative expression levels of the RNA complement of a cancerous cell relative to a normal (i.e., non-cancerous) cell. Because of the processing that may take place in transforming the initial RNA transcript into the final mRNA, the sequences disclosed herein may represent less than the full genomic sequence. They may also represent sequences derived from ribosomal and transfer RNAs. Consequently, the genes present in the cell (and representing the genomic sequences) and the sequences disclosed herein, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous or normal cells used to determine relative levels of expression because they represent the same sequences or are complementary to RNAs encoded by these genes. Such genes also include different alleles and splice variants that may occur in the cells used in the processes of the invention.

The genes of the invention “correspond to” TRIP13 (or a polynucleotide having a sequence of SEQ ID NO: 1-6) if the gene encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a polynucleotide having the indicated sequence. In addition, genes including sequences at least 90% identical to a sequence selected from SEQ ID NO: 1-6, preferably at least about 95% identical to such a sequence, more preferably at least about 98% identical to such sequence and most preferably comprising such sequence are specifically contemplated by all of the processes of the present invention as being genes that correspond to these sequences. In addition, sequences encoding the same proteins as any of these sequences, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within any of the sequences of SEQ ID NO: 1-6.

The present invention also finds use as a means of diagnosing the presence of cancer in a patient, as where a sample of cancerous tissues or cells, or tissues or cells suspected of being cancerous. For such purposes, and in accordance with the disclosure elsewhere herein, such diagnosis is based on the detection of elevated expression or amplification, such as elevated copy number, of one or more of the genes identified according to the invention. Such elevated expression can be determined by any of the means described herein.

In one such embodiment, the elevated expression, as compared to normal cells and/or tissues of the same organ, is determined by measuring the relative rates of transcription of RNA, such as by production of corresponding cDNAs and then analyzing the resulting DNA using probes developed from the gene sequences of SEQ ID NO: 1-6. The levels of cDNA produced by use of reverse transcriptase with the full RNA complement of a cell suspected of being cancerous produces a corresponding amount of cDNA that can then be amplified using polymerase chain reaction, or some other means, such as rolling circle amplification, to determine the relative levels of resulting cDNA and, thereby, the relative levels of gene expression.

For RNA analysis, the latter may be isolated from samples in a variety of ways, including lysis and denaturation with a phenolic solution containing a chaotropic agent, (e.g., triazol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies. Steady state RNA levels for a given type of cell or tissue may have to be ascertained prior to employment of the processes of the invention but such is well within the skill of those in the art and will not be further described in detail herein.

Alternatively, increased expression, such as increased copy number, may be determined for the genes present in a cancerous cell, or a cell suspected of being cancerous, by using the nucleotides sequences of SEQ ID NO: 1-6, as a means of generating probes for the DNAs present in the cells to be examined. Thus, the DNA of such cells may be extracted and probed using the sequences disclosed herein for the presence in the genomes of such cells of increased amounts of one or more of the genes of the invention. For example, where a cancer-related, or cancer-linked, gene as disclosed herein is found to be present in multiple copies within the genome of a cell, even where it may not be actively being overexpressed at the time of such determination, this may be indicative of at least a disposition toward developing cancer at a subsequent time.

In accordance with the foregoing, the presence of such multiple copies of a gene, or genes, as disclosed herein may be determined using northern or southern blotting and employing the sequences of SEQ ID NO: 1-6 to develop probes for this purpose. Such probes may be composed of DNA or RNA and may advantageously be comprised of a contiguous stretch of nucleotide residues matching, or complementary to, a sequence of SEQ ID NO: 1-6. Such probes will most usefully comprise a contiguous stretch of at least 15, preferably at least 30, more preferably at least 50, most preferably at least 80, and especially at least 100, even 200 residues, derived from one or more of the sequences of SEQ ID NO: 1-6. Thus, where a single probe binds multiple times to the genome of a sample of cells that are cancerous, or are suspected of being cancerous, or predisposed to become cancerous, whereas binding of the same probe to a similar amount of DNA derived from the genome of otherwise non-cancerous cells of the same organ or tissue results in observably less binding, this is indicative of the presence of multiple copies of a gene comprising, or corresponding to, the sequence of SEQ ID NO: 1-6 from which the probe sequenced was derived.

Increased expression may also be determined using agents that selectively bind to, and thereby detect, the presence of expression products of the genes disclosed herein. For example, an antibody, possibly a suitably labeled antibody, such as where the antibody is bound to a fluorescent or radiolabel, may be generated against one of the polypeptides comprising a sequence of SEQ ID NO: 7-11, and said antibody will then react with, binding either selectively or specifically, to a polypeptide encoded by one of the genes that corresponds to a sequence disclosed herein; Such antibody binding, especially relative extent of such binding in samples derived from suspected cancerous, as opposed to otherwise non-cancerous, cells and tissues, can then be used as a measure of the extent of expression, or over-expression, of the cancer-related genes identified herein. Thus, the genes identified herein as being over-expressed in cancerous cells and tissues may be over-expressed due to increased copy number, or due to over-transcription, such as where the over-expression is due to over-production of a transcription factor that activates the gene and leads to repeated binding of RNA polymerase, thereby generating large than normal amounts of RNA transcripts, which are subsequently translated into polypeptides, such as the polypeptides comprising amino acid sequences of SEQ ID NO: 7-11. Such analysis provides an additional means of ascertaining the expression of the genes identified according to the invention and thereby determining the presence of a cancerous state in a sample derived from a patient to be tested, of the predisposition to develop cancer at a subsequent time in said patient.

In employing the methods of the invention, it should be borne in mind that gene expression indicative of a cancerous state need not be characteristic of every cell found to be cancerous. Thus, the methods disclosed herein are useful for detecting the presence of a cancerous condition within a tissue where less than all cells exhibit the complete pattern of over-expression. For example, a set of selected genes, comprising sequences homologous under stringent conditions, or at least 90%, preferably 95%, identical to at least one of the sequences of SEQ ID NO: 1-8, may be found, using appropriate probes, either DNA or RNA, to be present in as little as 60% of cells derived from a sample of tumorous, or malignant, tissue while being absent from as much as 60% of cells derived from corresponding non-cancerous, or otherwise normal, tissue (and thus being present in as much as 40% of such normal tissue cells). In a preferred embodiment, such gene pattern is found to be present in at least 70% of cells drawn from a cancerous tissue and absent from at least 70% of a corresponding normal, non-cancerous, tissue sample. In an especially preferred embodiment, such gene pattern is found to be present in at least 80% of cells drawn from a cancerous tissue and absent from at least 80% of a corresponding normal, non-cancerous, tissue sample. In a most preferred embodiment, such gene pattern is found to be present in at least 90% of cells drawn from a cancerous tissue and absent from at least 90% of a corresponding normal, non-cancerous, tissue sample. In an additional embodiment, such gene pattern is found to be present in at least 100% of cells drawn from a cancerous tissue and absent from at least 100% of a corresponding normal, non-cancerous, tissue sample, although the latter embodiment may represent a rare occurrence.

In an additional aspect, the present invention relates to a method for determining a cancer initiating or facilitating gene comprising contacting a cell expressing a test gene (i.e., a gene whose status as a cancer initiating or facilitating gene is to be determined) with an agent that decreases the expression of a gene that encodes an RNA at least 90%, preferably 95%, identical to an RNA encoded by (i.e., a gene corresponding to) a polynucleotide comprising, or having, a sequence selected from the group consisting of SEQ ID NO: 1-6 and detecting a decrease in expression of said test gene compared to when said agent is not present, thereby identifying said test gene as being a cancer initiating or facilitating gene. Such genes may, of course, be oncogenes and said decrease in expression may be due to a decrease in copy number of said gene in said cell or a cell derived from said cell, such as where copy number is reduced in the cells formed by replication of such cells.

Thus, some or all of the sequences disclosed herein as corresponding to SEQ ID NO: 1-6 are found to play a direct role in the initiation or progression of cancer or even other diseases and disease processes. Because changes in expression of these genes (up-regulation) are linked to the disease state (i.e. cancer), the change in expression may contribute to the initiation or progression of the disease. For example, if a gene that is up-regulated is an oncogene such a gene provides for a means of screening: for small molecule therapeutics beyond screens based upon expression output alone. For example, genes that display up-regulation in cancer and whose elevated expression contributes to initiation or progression of disease represent targets in screens for small molecules that inhibit or block their function. Examples include, but are not be limited to, kinase inhibition, cellular proliferation, substrate analogs that block the active site of protein targets, etc.

It should be noted that there are a variety of different contexts in which genes have been evaluated as being involved in the cancerous process. Thus, some genes may be oncogenes and encode proteins that are directly involved in the cancerous process and thereby promote the occurrence of cancer in an animal. Other genes may simply be involved either directly or indirectly in the cancerous process or condition and may serve in an ancillary capacity with respect to the cancerous state. All such types of genes are deemed within those to be determined in accordance with the methods of the invention as disclosed herein where expression of such genes is modulated by an agent identified by the screening methods of the invention. Thus, the gene determined by said method of the invention may be an oncogene, or the gene determined by said process may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

In accordance with the foregoing, the method of the present invention includes cancer modulating agents that are themselves either polypeptides, or small chemical entities, that affect the cancerous process, including initiation, suppression or facilitation of tumor growth, either in vivo or ex vivo. Such agents may also be antibodies that react with one or more of the polypeptides of SEQ ID NO: 7-11.

In keeping with the disclosure herein, the present invention also relates to a method for treating cancer comprising contacting a cancerous cell with an agent having activity against an expression product encoded by a variant of TRIP13 or, alternatively, a gene corresponding to a polynucleotide that comprises a nucleotide sequence selected from SEQ ID NO: 1-6, such as where such expression product is one the polypeptides of SEQ ID NO: 7-11 that are encoded by the polynucleotides of SEQ ID NO: 1-6.

The methods of the present invention include embodiments of the above-recited process wherein said cancer cell is contacted in vivo as well as ex vivo, preferably wherein said agent comprises a portion, or is part of an overall molecular structure, having affinity for said expression product. In one such embodiment, said portion having affinity for said expression product is an antibody.

The present invention also relates to a method for diagnosing cancer comprising contacting a cancerous cell with an agent having affinity for an expression product of a gene corresponding to a polynucleotide comprising a nucleotide sequence of SEQ ID NO: 1-6 in an amount effective to cause a reduction in cancerous activity of said cell. In a preferred embodiment, the expression product is a polypeptide, most preferably a polypeptide that comprises an amino acid sequence of SEQ ID NO: 7-11. In one example of such embodiment, the detecting agent is an antibody, preferably one specific for a polypeptide having an amino acid sequence of SEQ ID NO: 7-11.

In one embodiment of the present invention, a chemical agent, such as a protein or other polypeptide, is joined to an agent, such as an antibody, having affinity for an expression product of a cancerous cell, such as a polypeptide or protein encoded by, a gene related to the cancerous process, especially a gene sequence corresponding to one of the cDNA sequences of SEQ ID NO: 1-6. In a specific embodiment, said expression product, preferably a polypeptide having one of SEQ ID NO: 7-11 as amino acid sequence, acts as a diagnostic and/or therapeutic target for the affinity portion of said anticancer agent and where, after binding of the affinity portion of such agent to the expression product, the anti-cancer portion of said agent acts against said expression product so as to neutralize its effects in initiating, facilitating or promoting tumor formation and/or growth. In a separate embodiment of the present invention, binding of the agent to said expression product may, without more, have the effect of deterring cancer promotion, facilitation or growth, especially where the presence of said expression product is related, either intimately or only in an ancillary manner, to the development and growth of a tumor. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

Many cancers contain chromosomal rearrangements, which typically represent translocations, amplifications, or deletions of specific regions of genomic DNA. A recurrent chromosomal rearrangement that is associated with a specific stage and type of cancer always affects a gene (or possibly genes) that play a direct and critical role in the initiation or progression of the disease. Many of the known oncogenes or tumor suppressor genes that play direct roles in cancer have either been initially identified based upon their positional cloning from a recurrent chromosomal rearrangement or have been demonstrated to fall within a rearrangement subsequent to their cloning by other methods. In all cases, such genes display amplification at both the level of DNA copy number and at the level of transcriptional expression at the mRNA level.

The present invention also relates to a method for determining functionally related genes comprising contacting one or more gene sequences corresponding to the cDNAs of SEQ ID NO: 1-6 with an agent that modulates expression of more than one gene in such group and thereby determining a subset of genes of said group.

In accordance with the present invention, said functionally related genes are genes modulating the same metabolic pathway or said genes are genes encoding functionally related polypeptides. In one such embodiment, said genes are genes whose expression is modulated by the same transcriptional activator or enhancer sequence, especially where said transcriptional activator or enhancer increases, or otherwise modulates, the activity of a gene corresponding to a cDNA of SEQ ID NO: 1-6.

The present invention also relates to a process that comprises a method for producing a product comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accordance with the foregoing, the present invention further relates to a method for producing test data with respect to the anti-neoplastic activity of a compound comprising:

(a) contacting a compound with a cell that expresses at least one gene corresponding to a polynucleotide comprising a nucleotide sequence selected from SEQ ID NO: 1-6 or that encodes a polypeptide having an amino acid sequence of SEQ ID NO: 7-1 land under conditions promoting said expression; and

(b) detecting a change in expression of said gene compared to expression when said contacting does not occur,

(c) producing test data with respect to the gene modulating activity of said compound based on a change in the expression of the determined gene, or genes, whose expression is otherwise elevated in a non-cancerous cell over that in a cancerous cell and a decrease in the expression of the determined gene, or genes whose expression is otherwise increased in a cancerous cell over that in a non-cancerous cell indicating anti-neoplastic activity.

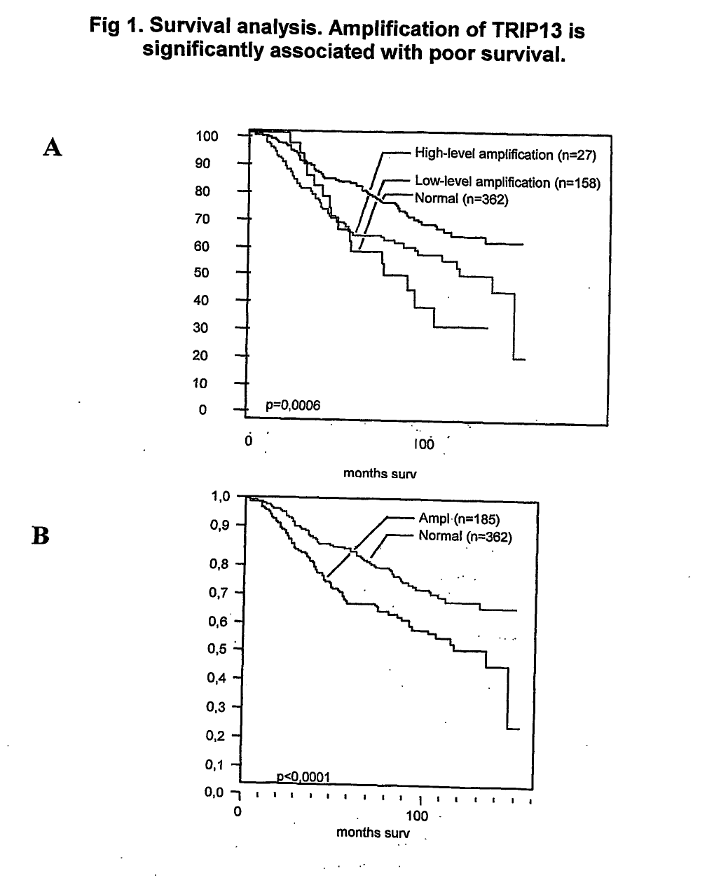

In another embodiment, the present invention provides a method for monitoring the progress of a cancer treatment, such as where the methods of the invention permit a determination that a given course of cancer therapy is or is not proving effective because of an increased or decreased expression of a gene, or genes, disclosed herein. For example, where there is an increased or decreased copy number of one or more of the genes corresponding to SEQ ID NO: 1-6 monitoring of such genes can predict success or failure of a course of therapy, such as chemotherapy, or predict the likelihood of a relapse based on elevated activity or expression of one or more of these genes following such course of therapy. Thus, TRIP can be used as a probe at diagnosis to determine the course of the disease, such as prospects for survival or relapse. The value of using TRIP for such prognosis determination is demonstrated by the data presented in FIG. 1 as further described in Example 2.

In accordance with the foregoing, the present invention contemplates a method for determining the progress of a treatment for cancer in a patient afflicted with cancer, following commencement of a cancer treatment on said patient, comprising:

(a) determining in said patient a change in expression of one or more genes corresponding to a polynucleotide comprising a nucleotide sequence of SEQ ID NO: 1-6 and under conditions promoting expression of said one or more genes; and

(b) detecting a change in expression of said gene compared to expression of said one or more determined genes prior to commencement of said cancer treatment;

thereby determining the progress of said treatment

In a preferred embodiment, the detected change in expression is a decrease in expression. In another preferred embodiment, the cancer treatment is treatment with a chemotherapeutic agent, especially an agent that modulates, preferably decreases, expression of a gene identified herein, such as where said agent was first identified as having anti-neoplastic activity using a method of the invention. Thus, in accordance with this aspect of the present invention, a patient, or even a research animal, such as a mouse, rat, rabbit or primate, afflicted with cancer, including a cancer induced for research purposes, is introduced to a cancer treatment regimen, such as administration of an anti-cancer agent, including one first identified as having anti-neoplastic activity by one or more of the screening methods disclosed herein. The progress and success or failure of such treatment is subsequently ascertained by determining the subsequent expression of one or more, including 2 or 3, or even all of the genes identified herein, following said treatment. In a preferred embodiment, a treatment that reduces said expression is deemed advantageous and may then be the basis for continuing said treatment. The methods of the invention thereby provide a means of continually monitoring the success of the treatment and evaluating both the need, and desirability, of continuing said treatment. In addition, more than one said treatment may be administered simultaneously without diminishing the value of the methods of the invention in determining the overall success of such combined treatment. Thus, more than one said anti-neoplastic agent may be administered to the same patient and overall effectiveness ascertained by the recited methods.

In accordance with the foregoing, the present invention also contemplates a method for determining survival prognosis of a patient afflicted with cancer, preferably breast cancer, comprising determining in said patient a change in expression of a TRIP13 gene versus a person not so afflicted wherein amplification of TRIP13 in said patient indicates a poor prognosis for survival of said patient. In one preferred embodiment, said TRIP13 gene corresponds to a polynucleotide comprising a nucleotide sequence selected from SEQ ID NO: 1-6 or that encodes a polypeptide having an amino acid sequence of SEQ ID NO: 7-11.

The present invention is also drawn to a method for determining the likelihood of survival of a patient afflicted with cancer, such as breast cancer, following commencement of a cancer treatment on said patient, comprising determining in said patient a change in expression of a TRIP13 gene following an anti-cancer treatment compared to such expression prior to commencement of said treatment, wherein a decrease in expression indicates likelihood of survival. In one preferred embodiment thereof, the TRIP13 corresponds to a polynucleotide comprising a nucleotide sequence selected from SEQ ID NO: 1-6 or that encodes a polypeptide having an amino acid sequence of SEQ ID NO: 7-11

The present invention also presents a method for diagnosing cancer, especially breast cancer, comprising contacting a cancerous cell with an agent having affinity for an expression product of a TRIP13 gene in an amount effective to cause a reduction in cancerous activity of said cell. In a preferred embodiment thereof, the TRIP13 gene corresponds to a polynucleotide comprising a nucleotide sequence selected from SEQ ID NO: 1-6 or that encodes a polypeptide having an amino acid sequence of SEQ ID NO: 7-11

In a preferred embodiment, the detected change in expression is a decrease in expression and said determined gene, or genes, may include 2, 3, 5, or all of the genes described herein. Thus, the methods of the invention may be utilized as a means for compiling cancer survival statistics following one or more, possibly combined, treatments for cancer as in keeping with the other methods disclosed herein.

The genes identified herein also offer themselves as pharmacodynamic markers (or as pharmacogenetic and/or surrogate markers), such as for patient profiling prior to clinical trials and/or targeted therapies, including combination treatments, resulting from the identification of these genes as valid gene targets for chemotherapy based on the screening procedures of the invention. In one embodiment thereof, the likelihood of success of a cancer treatment with a selected chemotherapeutic agent may be based on the fact that such agent has been determined to have expression modulating activity with one or more genes identified herein, especially where said genes have been identified as showing elevated expression levels in samples from a prospective patient afflicted with cancer. Methods described elsewhere herein for determining cancerous status of a cell may find ready use in such evaluations.

Such methods not only facilitate detection of the cancer but also permit stratification and/or selection of patients that are likely to respond or be refractory to treatment, thereby allowing more reliable decisions on specific treatment options based on levels of amplification and/or expression of Trip13 in such individuals. Thus, treatment becomes more acceptable and personal to the patient.

In any of the forgoing methods, the expression may be determined by determining a change in production of a polypeptide, preferably one that has an amino acid sequence selected from SEQ ID NO: 7-11. In one preferred embodiment, the production of said polypeptide is determined using an antibody that binds to said polypeptide, most preferably an antibody specific for a polypeptide having an amino acid sequence of SEQ ID NO: 7-11. Thus, antibodies find use in these methods as a means for detecting a protein in tissue, in situ and/or in vitro, as a marker for diagnosis and/or prognosis and/or treatment and/or to follow the course of treatment.

In any of the methods of the invention, said antibody may be polyclonal or monoclonal, or recombinant, and may include synthetic antibodies produced by polypeptide synthesis of the chains of the antibody. Thus, the method of producing the antibody, or antibodies, useful in the methods of the invention is non-limiting. In some embodiments, more than one antibody may be used to detect a single polypeptide, or a single antibody may be used to detect multiple polypeptides. In one example, multiple antibodies may be used to detect multiple polypeptides. The number of different antibodies used, and the number of different polypeptides detected, is likewise non-limiting.

It should be cautioned that, in carrying out the procedures of the present invention as disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art.

### EXAMPLE 1

### Gene Expression Analysis

Cancerous cells that over-express one or more of the genes selected from those that correspond to SEQ ID NO: 1-6 are grown to a density of 105 cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1×PCR buffer, RNAsin, reverse transcriptase, nucleoside triphosphates, amplitaq gold, tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perlin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference genes is then calculated and a relative expression value determined for all of the samples used. This procedure is then repeated for each of the target genes in a given signature, or characteristic, set and the relative expression ratios for each pair of genes is determined (i.e., a ratio of expression is determined for each target gene versus each of the other genes for which expression is measured, where each gene's absolute expression is determined relative to the reference gene for each compound or chemical agent, to be screened). The samples are then scored and ranked according to the degree of alteration of the expression profile in the treated samples relative to the control. The overall expression of the set of genes relative to the controls, as modulated by one chemical agent relative to another, is also ascertained. Chemical agents having the most effect on a given gene, or set of genes, are considered the most anti-neoplastic.

### EXAMPLE 2

### Analysis of TRIP 13 Amplification Status Using BAC FISH

Trip 13 gene amplification frequency and clinical significance in breast cancers was established using BAC probes derived from the sequences disclosed herein.

The samples were probed using Fluorescence in situ hybridization (FISH). In a typical experiment, 1 or 2 μg of probe were labeled by standard Nick-translation procedures. Hybridization was performed for 48 hrs at 37° C. in a moist chamber. Material used was a Breast Prognostic Array (Edition 2a—Diomeda Biosciences Inc.).

TABLE 1
Histological subtypes of the tissues in the tumor microarray.

| | No. of | | |
|---|---|---|---|
| | Cases | No. of Cases with | |
| Histological subtype | (amp) | successful FISH | Frequency |
| Ductal carcinoma | 19 | 364 | 5.2% |
| Cribriform carcinoma | 2 | 26 | 7.7% |
| Apocrine carcinoma | 2 | 8 | 25% |
| Lobular carcinoma | 1 | 77 | 1.3% |
| Medullary carcinoma | 1 | 9 | 11.1% |
| Papillary carcinoma | 1 | 13 | 3.3% |
| Clear cell carcinoma | 1 | 5 | 20% |

TABLE 2
Breakdown of tissues on the TMA
by Stage, Grade and Metastases

| | | No. of cases | No. of cases | |
|---|---|---|---|---|
| | | with amplified | with normal | |
| FEATURE | | TRIP13 | copy number | |
| Tumor stage | pT1 | 7(4.1%) | 170 | |
| | pT2 | 13(4.7%) | 274 | |
| | pT3 | 3(7.1%) | 42 | |
| | pT4 | 3(5.1%) | 59 | |
| Tumor grade | G1 | 2(1.2%) | 166 | |
| | G2 | 9(4.0%) | 224 | |
| | G3 | 16(10.1%) | 158 | |
| Nodal metastases | pN0 | 9(3.5%) | 257 | |
| | pN1 | 12 (6.1%) | 197 | |
| | pN2 | 2(6.9%) | 29 | |
| Age (median) | | 64 years | 64 | years |
| Tumor size (median) | | 25 mm | 25 | mm |

p = 0.02.
Contingency table, chi-square test.

Here, probes were used that span the region on 5p15.33 that harbors the gene trip13.

The amplification status of TRIP13 was also examined on a formalin-fixed tissue microarray (TMA) consisting of 785 breast cancer samples by FISH. A BAC probe from the region of the TRIP13 gene (test probe) and a reference probe consisting of a BAC probe mapping to the sub-centromeric region of chromosome 5 were fluorescently labeled and individually hybridized to a Breast Prognostic Tissue MicroArray. (TMA) (FIG. 1). The test and reference probes could only be evaluated in 547 of the 785 breast samples in the TMA. The BAC from the TRIP13 region exhibited high-level amplification (>3 fold) in 5% (27 of 547) of cases, and low-level amplification (2 to 3 fold) in 29% (158 of 547) of breast cancer cases. TRIP13. amplification was found more frequently in ductal carcinomas (5.2%, 19 of 364 cases) than in lobular carcinomas (1.3%, 1 of 77 cases), and was significantly correlated with high-grade tumors (G1=1.2%, G2=4.0%, G3=10.1%; P=0.02,) but not with tumor stage, size or nodal metastasis. This result is summarised in Table 3.

TABLE 3
Breast Tumor TMA FISH with TRIP-13.

| Copy Number | No. of Samples | Percent of Total |
|---|---|---|
| High-level amplified (>3 fold) | 27 | 5% |
| Low-level amplified (2-3 fold) | 158 | 29% |
| Normal | 362 | 66% |

TRIP13 amplification was an independent prognostic marker in breast cancer, as shown by determination of the correlation between TRIP13 amplification and survival outcome for cases represented on the TMA. Analysis of patient clinical data linked to the samples on the TMA showed that TRIP13 amplification correlated with poor survival in breast cancer patients (P=0.0001). Furthermore, patients with high-level amplification had the worst survival rate (P=0.0006). TRIP13 amplification showed significant association with poor prognosis in the absence (p<0.02 amplified, P,0.0002 positive) and independent of HER2/NEU over-expression (P<0.002 amplified, P<0.0002 positive). TRIP13 also shows significant association with poor prognosis additive with HER2/NEU, compared to when neither of these genes are affected (P<0.02 amplified, P<0.00001 positive). Moreover, TRIP13 was also found to be highly expressed in a subset of ER negative breast cancer samples in the GX2000™ database.

For HER2/NEU, the Hercept test measures the levels of DNA amplification of HER2/NEU in biopsy, using fluorescent hybridization. In accordance with the present invention, TRIP13 is an independent prognostic indicator of survival in breast cancer such that increased expression of TRIP13 leads to poor survival expectation irrespective of HER2/NEU. Thus, for purposes of treatment, failure to reduce TRIP13 expression leads to poor survival even if HER2/NEU expression is reduced. Thus, the present invention presents two aspects of breast cancer survival: first is that increased expression of TRIP13 predicts poor survival even if HER2/NEU is not amplified and second, treatments that reduce HER2/NEU expression levels but that do not reduce TRIP13 expression are expected to result in a poor prognosis for the patient. For example, the aforementioned Hercept test is used to check HER2/NEU amplification in patients' biopsies before commencing treatment with herceptin. A similar test can be performed for TRIP13 amplification, independent of HER2/NEU, prior to administering treatment for TRIP13 amplification, which is specifically contemplated by the present invention.

### EXAMPLE 3

### Gene Co-Expression

Genes co-expressed with TRIP13 were analyzed in a set of 151 malignant breast tumor samples on the Affymetrix HG_U133 chipset. Comparison of samples with high levels of TRIP13 to those with low levels of TRIP13 uncovered a group of genes that had expression levels. Several known genes were found to be significantly (p<0.0001) co-expressed with TRIP13, the highest correlation being with genes involved in mitotic spindle assembly and kineticore function (CENPA, KNSL5, TPX2, PRC1, ZWINT, BUB1, SMC4L1, MCM6, DLG7). Other co-expressed genes included those involved in G2/M transition (Cyclin A2, Cyclin B1, Cyclin B2, Cdc2, Cdc28 kinase 1 and 2) and in chromosome structure and maintenance (STK12, STK15, PLK, CDC45L, MCM6, CHK1 and MAD2). This data corroborates the role of TRIP13 in cell cycle regulation and maintenance of chromosome integrity.

### EXAMPLE 4

### Phenotypic Effect of TRIP13 Expression

RNA interference (RNAi) was used to examine the effect of silencing the expression of TRIP13 mRNA in a tumor cell line. MCF7 cells, which express TRIP13 mRNA at a moderate level, were transfected with siRNAs against TRIP13 (TRIP13i), and a negative control (TRIP13mi) containing a two base pair mismatch. TRIP13i resulted in a 90% reduction of the steady state level of TRIP13 mRNA compared to TRIP13Mi treated cells. We measured the levels of DNA synthesis 48 hrs after RNAi treatment and found a 30-40% drop in BrdU incorporation suggesting TRIP13 silencing had an effect on cell cycle progression. The number of mitotic cells present after siRNA treatment was determined by staining with an antibody specific for mitotic cells. A significant increase in mitotic cells was found in the TRIP13i treated cells compared to TRIP13mi treatment. This evidence suggests that TRIP13 expression is required for progression through the cell cycle, and it's inhibition causes cells to accumulate in mitosis (G2/M arrest).

What is claimed is:

1. A method for identifying a TRIP13 gene modulating agent, comprising:
(a) contacting a test compound with a cell that expresses a TRIP 13 gene; and
(b) determining a change in expression of said gene as a result of said contacting, wherein a change in said determined expression indicates gene modulation,
thereby identifying said test compound as a gene modulating agent.

2. The method of claim 1 wherein said change in expression is a decrease in expression.

3. The method of claim 2 wherein said decrease in expression is a decrease in copy number of the gene.

4. The method of claim 1 wherein said TRIP13 gene corresponds to a polynucleotide comprising a nucleotide sequence selected from SEQ ID NO: 1-6.

5. The method of claim 1 wherein said gene comprises a nucleotide sequence that is a splice variant of TRIP13.

6. The method of claim 1 wherein the cell expressing said gene is a recombinant cell engineered to express a splice variant of TRIP13.

7. The method of claim 1 wherein said change in expression is a decrease in the synthesis of an RNA encoded by said gene.

8. The method of claim 1 wherein said change in expression is a decrease in the synthesis of a polypeptide encoded by said gene.

9. The method of claim 8 wherein said polypeptide is a member selected from the group consisting of the polypeptides having amino acid sequence of SEQ ID NO: 7-11.

10. A method for identifying an anti-neoplastic agent comprising contacting a cancerous cell with a compound found to have gene modulating activity in the method of claim 1 under conditions promoting the growth of said cell and detecting a change in the activity of said cancerous cell.

11. The method of claim 10 wherein said change in activity is a decrease in the rate of replication of said cancerous cell.

12. The method of claim 10 wherein said change in activity is a decrease in the total number of progeny cells that can be produced by said cancerous cell.

13. The method of claim 10 wherein said change in activity is a decrease in the number of times said cancerous cell can replicate.

14. The method of claim 10 wherein said change in activity is the death of said cancerous cell.

15. The method of claim 10 wherein said cancerous cell is a recombinant cell.

16. A method for detecting the cancerous status of a cell, comprising detecting elevated expression in said cell of at least one gene corresponding to a polynucleotide comprising a nucleotide sequence selected from SEQ ID NO: 1-6 whereby such elevated expression is indicative of cancerous status of the cell.

17. The method of claim 16 wherein said elevated expression is an elevated copy number of the gene.

18. The method of claim 16 wherein the gene comprises a sequence of SEQ ID NO: 1-6.

19. A method for detecting a cancer-linked gene comprising the steps of contacting a compound that decreases expression of a gene corresponding to a polynucleotide comprising a nucleotide sequence selected from SEQ ID NO: 1-6, or that encodes a polypeptide having an amino acid sequence of SEQ ID NO: 7-11, with a cell containing a gene to be tested and detecting a decrease in expression of said test gene thereby identifying said gene as a cancer-linked gene.

20. The method of claim 19 wherein the gene comprises a sequence of SEQ ID NO: 1-6.

21. A method for identifying an agent that modulates a TRIP13 polypeptide biological activity, comprising:
(a) contacting a test compound with a TRIP13 polypeptide; and
(b) determining a change in biological activity of said TRIP13 polypeptide as a result of said contacting,
wherein a change in said biological activity indicates modulation of TRIP13 biological activity,
thereby identifying said test compound as an agent that modulates TRIP13 biological activity.

22. The method of claim 21 wherein said determined change is a decrease in biological activity.

23. The method of claim 21 wherein said TRIP13 polypeptide is present in a cell.

24. The method of claim 23 wherein said cell is a mammalian cell.

25. The method of claim 23 wherein said cell has been engineered to contain a TRIP13 polypeptide.

26. The method of claim 21 wherein said TRIP13 polypeptide comprises an amino acid sequence selected from SEQ ID NO: 7, 8, 9, 10, 11 and 12.

27. The method of claim 21 wherein said TRIP13 polypeptide is immobilized on a solid support.

28. A method for detecting cancer or a disposition toward developing cancer comprising detecting in a sample from a patient an increase in expression of a gene corresponding to a polynucleotide comprising a nucleotide sequence selected from SEQ ID NO: 1-6 or that encodes a polypeptide having an amino acid sequence of SEQ ID NO: 7-11.

29. The method of claim 28 wherein said increase in expression is an increase in copy number of the gene.

30. The method of claim 28 wherein said gene comprises a nucleotide sequence of SEQ ID NO: 1-6.

31. A method for treating cancer comprising contacting a cancerous cell with an agent first identified as having gene modulating activity using the method of claim 1 and in an amount effective to cause a reduction in cancerous activity of said cell.

32. The method of claim 31 wherein said cancerous cell is contacted in vivo.

33. The method of claim 31 wherein said reduction in cancerous activity is a decrease in the rate of proliferation of said cancerous cell.

34. The method of claim 31 wherein said reduction in cancerous activity is the death of said cancerous cell.

35. The method of claim 31 wherein said cancer is a cancer of breast, colon, lung or prostate tissues.

36. A method for treating cancer comprising contacting a cancerous cell with an agent having affinity for an expression product of a gene corresponding to a polynucleotide comprising a nucleotide sequence of SEQ ID NO: 1-6 in an amount effective to cause a reduction in cancerous activity of said cell.

37. The method of claim 36 wherein said expression product is a polypeptide.

38. The method of claim 37 wherein said polypeptide comprises an amino acid sequence of SEQ ID NO: 7-11.

39. The method of claim 36 wherein said agent is an antibody.

40. A method for monitoring the progress of cancer therapy in a patient comprising monitoring in a patient undergoing cancer therapy the expression of a gene corresponding to a polynucleotide having a sequence of SEQ ID NO: 1-6 wherein a decrease in said expression is indicative of success of said cancer therapy.

41. The method of claim 40 wherein said gene comprises a sequence of SEQ ID NO: 1-6.

42. The method of claim 40 wherein said cancer therapy is chemotherapy.

43. The method of claim 40 wherein said cancer is a solid tumor, or a cancer of breast, colon, lung or prostate tissues.

44. A method for determining the likelihood of success of cancer therapy in a patient, comprising monitoring in a patient undergoing cancer therapy the expression of a gene corresponding to a polynucleotide, having a sequence of SEQ ID NO: 1-6 wherein a decrease in said expression prior to completion of said cancer therapy is indicative of a likelihood of success of said cancer therapy.

45. The method of claim 44 wherein said gene comprises a sequence of SEQ ID NO: 1-6.

46. A method for producing test data with respect to the anti-neoplastic activity of a compound comprising:
(a) contacting a compound with a cell that expresses at least one gene corresponding to a polynucleotide comprising a nucleotide sequence selected from SEQ ID NO: 1-6 or that encodes a polypeptide having an amino acid sequence selected from SEQ ID NO: 7-11; and
(b) determining a change in expression of said gene compared to expression when said contacting does not occur, (c) producing test data with respect to the gene modulating activity of said compound based on a change in the expression of the determined gene, or genes, whose expression is otherwise elevated in a non-cancerous cell over that in a cancerous cell and a decrease in the expression of the determined gene, or genes whose expression is otherwise increased in a cancerous cell over that in a non-cancerous cell indicating anti-neoplastic activity.

47. A method for determining the progress of a treatment for cancer in a patient afflicted therewith, following commencement of a cancer treatment on said patient, comprising:
(a) determining in said patient a change in expression of one or more genes corresponding to a polynucleotide comprising a nucleotide sequence selected from SEQ ID NO: 1-6 or that encodes a sequence selected from SEQ ID NO: 7-11 and under conditions promoting said expression; and
(b) determining a change in expression of said gene compared to expression of said, one or more determined genes prior to commencement of said cancer treatment;
thereby determining the progress of said treatment.

48. The method of claim 47 wherein the change in expression determined in (b) is a change in expression of more than one such gene.

49. The method of claim 45 wherein said production of a polypeptide is determined using an antibody that binds to said polypeptide.

50. The method of claim 47 wherein said antibody is specific for a polypeptide having an amino acid sequence of SEQ ID NO: 7-11.

51. A method for determining survival prognosis of a patient afflicted With cancer, comprising determining in said patient a change in expression of a TRIP13 gene versus a person not so afflicted wherein amplification of TRIP13 in said patient indicates a poor prognosis for survival of said patient.

52. The method of claim 51 wherein said cancer is breast cancer.

53. The method of claim 51 wherein said TRIP13 gene corresponds to a polynucleotide comprising a nucleotide sequence selected from SEQ ID NO: 1-6 or that encodes a polypeptide having an amino acid sequence of SEQ ID NO: 7-11.

54. A method for determining the likelihood of survival of a patient afflicted with cancer, following commencement of a cancer treatment on said patient, comprising determining in said patient a change in expression of a TRIP13 gene following an anti-cancer treatment compared to such expression prior to commencement of said treatment, wherein a decrease in expression indicates likelihood of survival.

55. The method of claim 54 wherein said cancer is breast cancer.

56. The method of claim 54 wherein said TRIP13 corresponds to a polynucleotide comprising a nucleotide sequence selected from SEQ ID NO: 1-6 or that encodes a polypeptide having an amino acid sequence of SEQ ID NO: 7-11

57. A method for diagnosing cancer comprising contacting a cancerous cell with an agent having affinity for an expression product of a TRIP13 gene in an amount effective to cause a reduction in cancerous activity of said cell.

58. The method of claim 57 wherein said agent is an antibody.

59. The method of claim 57 wherein said TRIP13 gene corresponds to a polynucleotide comprising a nucleotide sequence selected from SEQ ID NO: 1-6 or that encodes a polypeptide having an amino acid sequence of SEQ ID NO: 7-11
